(19) United States
(12) Patent Application Publication
(10) Pub. No.: US 2014/0031621 A1
(43) Pub. Date: Jan. 30, 2014

(54) CUTTING APPARATUS WITH IMAGE CAPTURE ARRANGEMENT FOR TREATING CARPAL TUNNEL SYNDROME

(71) Applicant: Jung-Tung Liu, Taichung City (TW)

(72) Inventor: Jung-Tung Liu, Taichung City (TW)

(21) Appl. No.: 13/557,553

(22) Filed: Jul. 25, 2012

(51) Int. Cl. A61B 1/00

(52) U.S. Cl. 600/103

(57) ABSTRACT

A cutting apparatus for treating carpal tunnel syndrome is provided with a hollow handle including a forward bent portion, a switch mounted on an outer surface, and a video screen pivotably secured to the outer surface; a transparent blade mount mounted with the bent portion and including an upper guide and a lower guide; a metal blade between the upper and lower guides; and an image capture assembly including a light source in the handle proximate the bent portion, a digital camera proximate the blade, and a microprocessor in the handle. The microprocessor is electrically connected to the video screen, the light source, and the digital camera respectively. The digital camera takes video of a target and records same as images. The microprocessor processes the images sent from the digital camera into image signals. The video screen receives the image signals and displays same as images.

### BACKGROUND OF THE INVENTION

1. Field of the Invention

The invention relates to surgical cutting apparatuses and more particularly to a cutting apparatus having an image capture arrangement for treating carpal tunnel syndrome.

2. Description of Related Art

Carpal tunnel syndrome (CTS) is an entrapment median neuropathy, causing paresthesia, pain, numbness, and other symptoms in the distribution of the median nerve due to its compression at the wrist in the carpal tunnel. The pathophysiology is not completely understood but can be considered compression of the median nerve traveling through the carpal tunnel. The main symptom of CTS is intermittent numbness of the thumb, index, long and radial half of the ring finger. The numbness often occurs at night, with the hypothesis that the wrists are held flexed during sleep. It is suggested that sleep positioning, such as sleeping on one's side, may be an associated factor. It can be relieved by wearing a wrist splint that prevents flexion. Pain in CTS is primarily numbness.

The only scientifically established disease modifying treatment is surgery to cut the transverse carpal ligament. Taiwan Utility Model Number M355694 discloses a knife for treating carpal tunnel syndrome as shown in FIG. 1. The integral knife comprises a handle, a blade, and a bent portion interconnecting the handle and the blade. The blade comprises a lower planar guide, an upper guide finger, a neck interconnecting the planar guide and the guide finger, a cutting edge formed on a forward curved notch of the neck. The planar guide is parallel to the guide finger but larger than the guide finger. A surgeon may make an incision in the skin on the wrist at about the level of the distal palmar crease.

However, a drawback has been found in the conventional knife for treating carpal tunnel syndrome. In detail, no light source, digital camera, and video screen are provided for the surgery. Thus, a precise positioning of the knife on the surgical site completely depends upon the experience and skill of the surgeon. This is not reliable. Thus, the need for improvement still exists.

### SUMMARY OF THE INVENTION

It is therefore one object of the invention to provide a cutting apparatus for treating carpal tunnel syndrome comprising a hollow handle comprising an interior space, a bent portion at a forward end of the handle, a switch mounted on an outer surface of the handle, and a video screen pivotably secured to the outer surface of the handle; a blade mount formed of transparent material and having a rear end mounted with the bent portion of the handle, the blade mount comprising an upper guide and a lower guide; a metal blade disposed between the upper guide and the lower guide and being perpendicular to each of the upper guide and the lower guide; and an image capture assembly comprising a light source disposed in the interior space proximate the bent portion, a digital camera disposed proximate the blade, and a microprocessor disposed in the interior space; wherein the microprocessor is electrically connected to the video screen, the light source, and the digital camera respectively; wherein the digital camera takes video of a target and records same as images; wherein the microprocessor processes the images sent from the digital camera into image signals; and wherein the video screen receives the image signals from the microprocessor and displays same as images.

The above and other objects, features and advantages of the invention will become apparent from the following detailed description taken with the accompanying drawings.

### BRIEF DESCRIPTION OF THE DRAWINGS

FIG. 1 is a perspective view of a conventional knife for treating carpal tunnel syndrome;

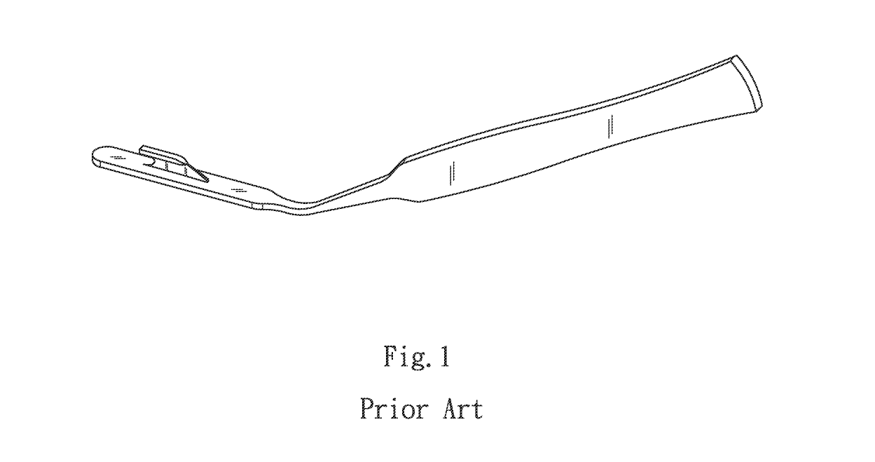

FIG. 2 is a perspective view of a cutting apparatus for treating carpal tunnel syndrome according to the invention, the Video screen being pivoted to a ready to watch position;

FIG. 3 is a view similar to FIG. 2, the Video screen being pivoted to an inoperative position;

FIG. 4 is a perspective view of the cutting apparatus for treating carpal tunnel syndrome, a first preferred embodiment of the invention shown without the provision of optical fibers;

FIG. 5 is a view similar to FIG. 4, a second preferred embodiment of the invention shown with the provision of optical fibers;

FIG. 6 is a longitudinal sectional view of the cutting apparatus shown in FIG. 5;

FIG. 7 schematically depicts the palm of the hand of a human being;

FIG. 8 is a perspective view of the cutting apparatus making an incision in the skin of the wrist at about the level of the distal palmar crease during surgery; and

FIG. 9 is a block diagram of an image capture procedure performed by the cutting apparatus during the surgery.

### DETAILED DESCRIPTION OF THE INVENTION

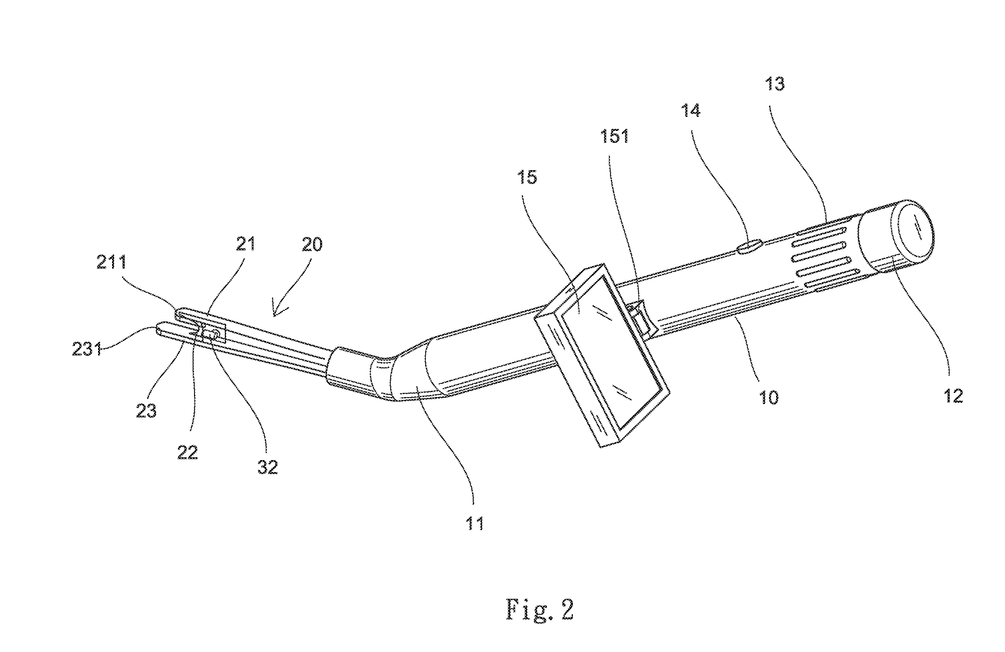

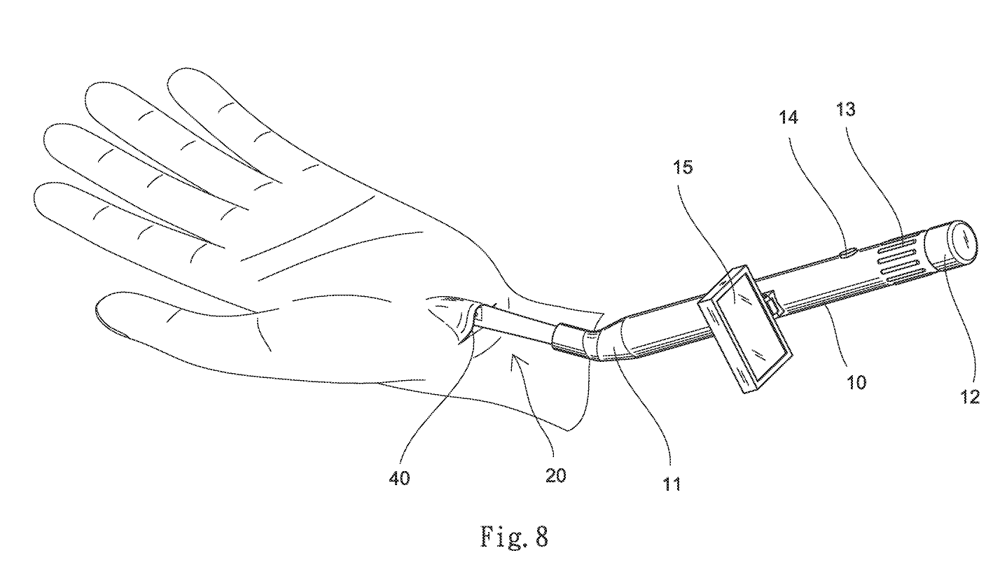

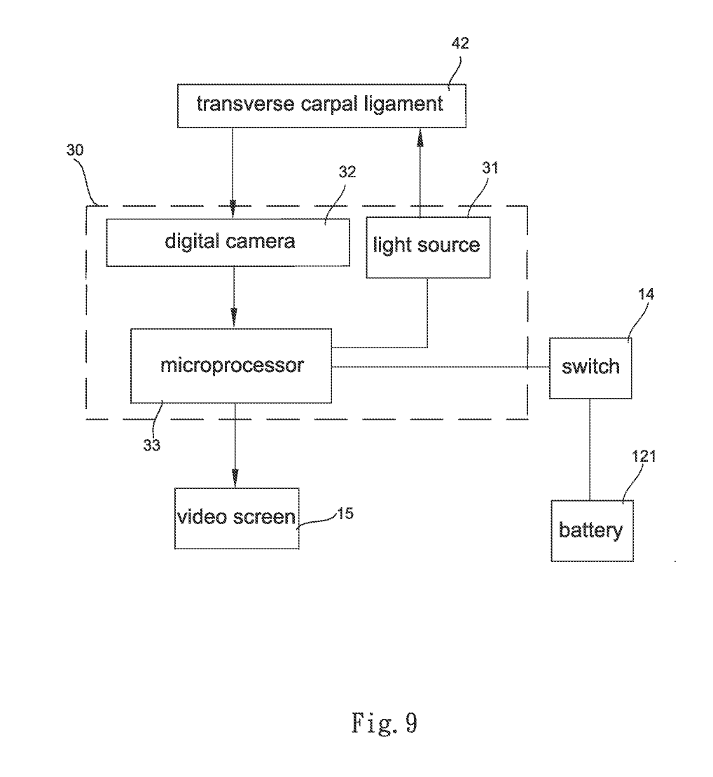

Referring to FIGS. 2 to 9, a cutting apparatus for treating carpal tunnel syndrome in accordance with the invention comprises a handle 10, a blade mount 20 provided on a forward end of the handle 10, and an image capture assembly 30. Each of the components will be as discussed in detail below.

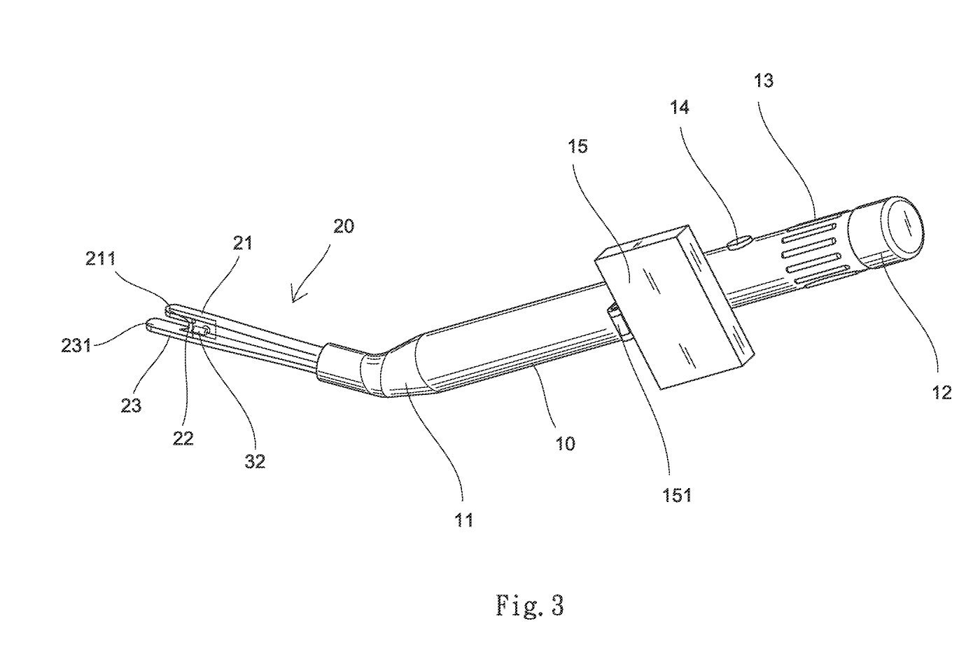

The handle 10 is an elongated, hollow cylinder and comprises an interior space 101, a bent portion 11 at a forward end for mounting with the blade mount 20, a releasable cap 12 on a rear end, a battery 121 provided in the space 101 proximate the cap 12, a plurality of parallel ribs (as a slip resistant member) 13 formed around an outer surface of the handle 10 proximate the cap 12, a switch 14 formed on the outer surface of the handle 10 proximate the ribs 13 and electrically connected to the battery 121, and a video screen 15 having a pivot 151 pivotably secured to the outer surface of the handle 10 and spaced from the switch 14. Thus, the video screen 15 can be disposed in either an inoperative position (see FIG. 3) or a ready to watch position during surgery (see FIG. 2).

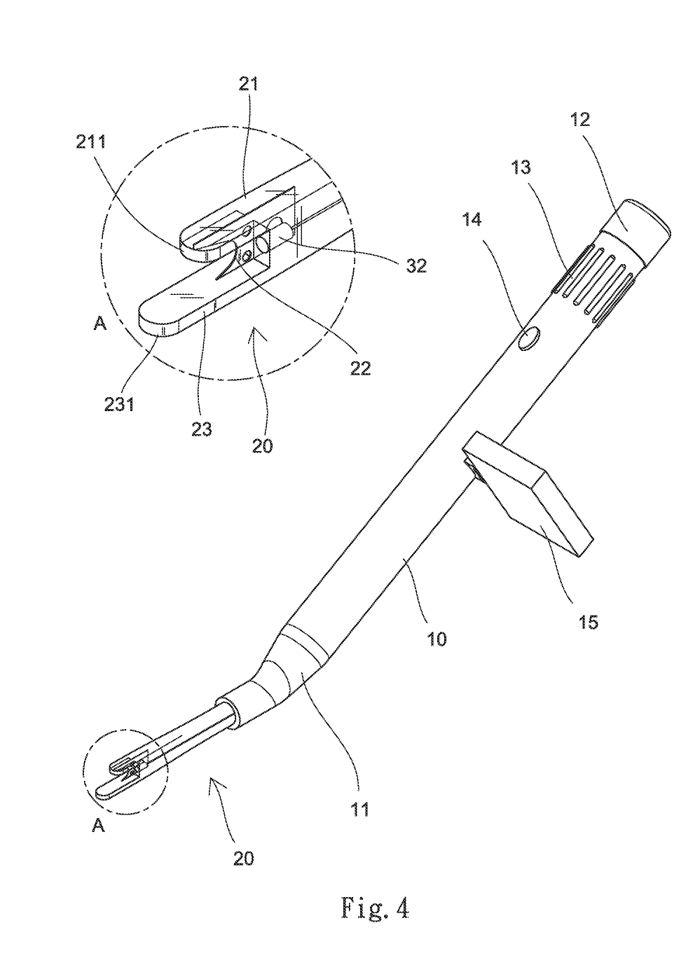

The blade mount 20 is made of transparent material with light guiding capability. The blade mount 20 has a rear end mounted with the bent portion 11 of the handle 10. In a first preferred embodiment of the invention (see FIG. 4), the blade mount 20 comprises a planar, upper guide 21 including a forward, curved light focusing end 211, and a planar, lower guide 23 spaced from the upper guide 21 and having a length and a width both greater than that of the upper guide 21, the lower guide 23 including a forward, curved light focusing end 231.

A curved blade 22 is made of metal and provided between the upper guide 21 and the lower guide 23. Further, the blade 22 is perpendicular to each of the upper guide 21 and the lower guide 23.

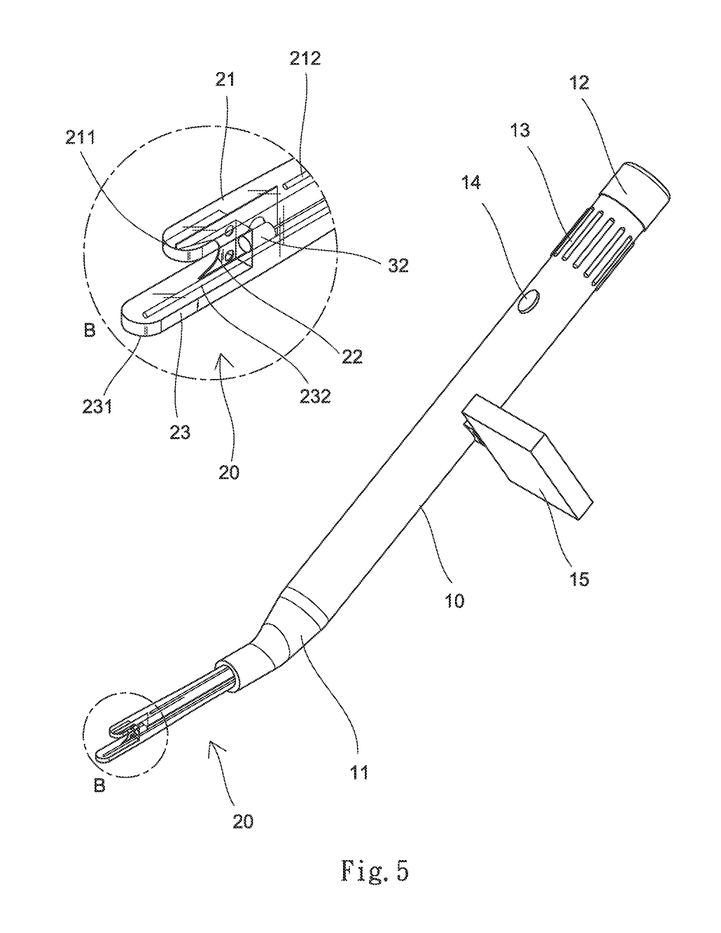

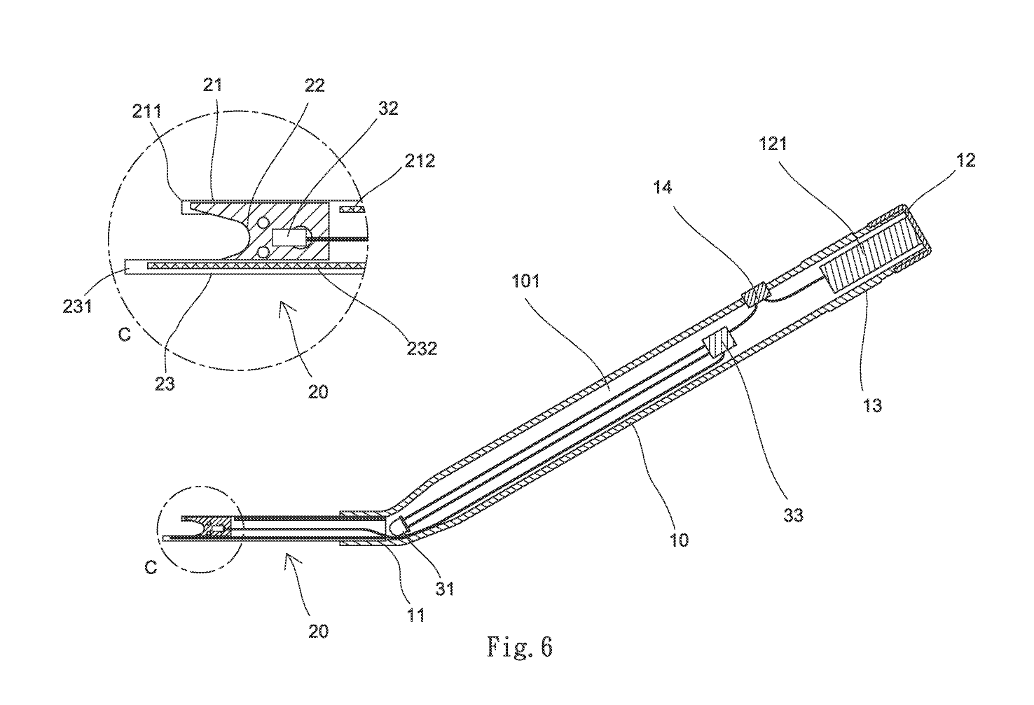

In a second preferred embodiment of the invention (see FIGS. 5 and 6), the blade mount 20 further comprises a first optical fiber 212 disposed from a rear end of the blade mount 20 to a position proximate the forward end of the upper guide 21, and a second optical fiber 232 disposed from a rear end of the blade mount 20 to a position proximate the forward end of the lower guide 23, the second optical fiber 232 being parallel to the first optical fiber 212. The provision of the first and second optical fibers 212, 232 can increase the light focusing capability of the forward end of the blade mount 20.

The image capture assembly 30 comprises a light source 31 provided in the space 101 proximate the bent portion 11, a digital camera 32 proximate the blade 22, and a microprocessor 33 provided in the space 101 proximate the switch 14, the microprocessor 33 electrically connected to the switch 14, the microprocessor 33 further electrically connected to the video screen 15, the light source 31 and the digital camera 32 respectively. The light source 31 is a light-emitting diode (LED) and is adapted to emit light toward the forward end of the blade mount 20. The digital camera 32, as commanded by the microprocessor 33, is capable of taking video of a target (as detailed later) by recording images of the target on its electronic image sensor. Further, the recorded images in the image sensor are processed by the microprocessor 33 and sent to the video screen 15 for display as detailed later.

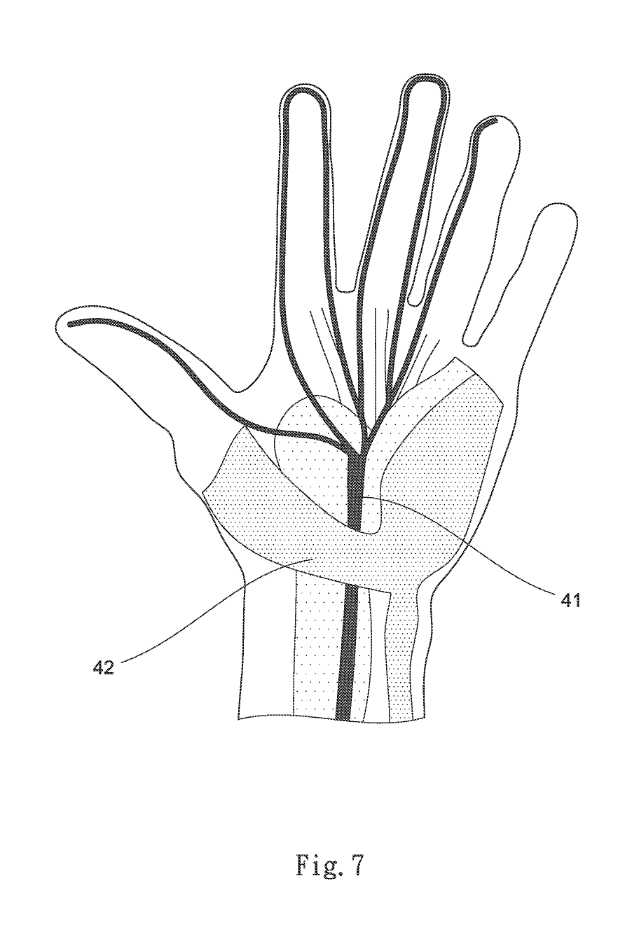

The operation of the cutting apparatus of the invention for treating CTS (i.e., releasing the transverse carpal ligament) in a surgery will be described by referring to FIGS. 7, 8 and 9 specifically. First, a surgeon turn on the switch 14 and thus all electronic and electrical components of the cutting apparatus are activated. Next, the surgeon may hold the handle 10 by grasping the ribs 13. Next, the surgeon may make an incision 40 in the skin on the wrist at about the level of the distal palmar crease and insert the blade 22 of the blade mount 20 through the incision 40. Light emitted by the light source 31 may travel to the light focusing ends 211, 231 and further focus on a target (i.e., the tissues suffering CTS in the palm). Images of the target are taken by the digital camera 32 and recorded on the image sensor. Next, the recorded images are sent to the microprocessor 33 for processing into image signals. Finally, the image signals are sent to the video screen 15 for display as images.

It is envisaged by the invention that the surgeon can clearly see the target (i.e., surgical site) by watching the video screen 15 during the surgery. Thus, the surgeon can correctly follow the progress of the surgery on the video screen 15 to obtain a clear view of the target. Therefore, the surgeon can precisely manipulate the blade mount 20 by pushing both the upper guide 21 and the lower guide 23 toward the target and bypassing the median nerve 41. Finally, the surgeon can push the blade 22 to cut the transverse carpal ligament 42 and successfully release same.

While the invention has been described in terms of preferred embodiments, those skilled in the art will recognize that the invention can be practiced with modifications within the spirit and scope of the appended claims.

What is claimed is:

1. A cutting apparatus for treating carpal tunnel syndrome comprising:
a hollow handle comprising an interior space, a bent portion at a forward end of the handle, a switch mounted on an outer surface of the handle, and a video screen pivotably secured to the outer surface of the handle;
a blade mount formed of transparent material and having a rear end mounted with the bent portion of the handle, the blade mount comprising an upper guide and a lower guide;
a metal blade disposed between the upper guide and the lower guide and being perpendicular to each of the upper guide and the lower guide; and
an image capture assembly comprising a light source disposed in the interior space proximate the bent portion, a digital camera disposed proximate the blade, and a microprocessor disposed in the interior space;
wherein the microprocessor is electrically connected to the video screen, the light source, and the digital camera respectively;
wherein the digital camera takes video of a target and records same as images;
wherein the microprocessor processes the images sent from the digital camera into image signals; and
wherein the video screen receives the image signals from the microprocessor and displays same as images.

2. The cutting apparatus of claim 1, wherein the handle further comprises a releasable cap on a rear end, a battery disposed in the interior space proximate the cap and electrically connected to the switch, and a plurality of parallel ribs formed around the outer surface of the handle proximate the cap.

3. The cutting apparatus of claim 1, wherein the upper guide is planar and includes a forward, curved light focusing end, and wherein the lower guide is planar and has a length and a width both greater than that of the upper guide, the lower guide including a forward, curved light focusing end.

4. The cutting apparatus of claim 1, wherein the blade mount further comprises a first optical fiber disposed from the rear end of the blade mount to a position proximate a forward end of the upper guide, and a second optical fiber disposed from the rear end of the blade mount to a position proximate a forward end of the lower guide.

5. The cutting apparatus of claim 2, wherein the light source is electrically connected to the switch and the battery via the microprocessor respectively, and wherein the light source emits light toward the blade mount.

6. The cutting apparatus of claim 1, wherein the digital camera is commanded by the microprocessor.

7. The cutting apparatus of claim 1, wherein the video screen is electrically connected to the microprocessor which is electrically connected to the switch and the battery respectively.
